(19) United States
(12) Patent Application Publication
(10) Pub. No.: US 2003/0219715 A1
(43) Pub. Date: Nov. 27, 2003

(54) Cell-based analysis of high throughput screening data for drug discovery

(76) Inventors: Raymond L H Lam, Oakville (CA); William J Welch, Waterloo (CA); Sidney Stanley Young, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/344,081

(22) PCT Filed: Aug. 9, 2001
(86) PCT No.: PCT/US01/25003

(51) Int. Cl. C12Q 1/00; G06F 19/00; G01N 33/48; G01N 33/50

(52) U.S. Cl. 435/4; 702/19

(57) ABSTRACT

A method of drug discovery in finding compounds with some desired activity for a chosen biological target that reduces reliance on searches of relatively large numbers of compounds. Rules are derived from a relatively small screening data set linking structural features to biological activity, which rules can be used to guide the selection of additional compounds for screening so that screening costs can be reduced as the total number of compounds screened is reduced. Properties of compounds are described numerically and said compounds are placed into small mathematical bins that comprise narrow ranges of the numerical descriptors. Through statistical analysis, it is then determined which combinations of bins describe regions of chemical space that are most likely to contain active compounds. Untested compounds that fall into these regions are then seen as good candidates for screening.

### FIELD OF THE INVENTION

In biological screening for drug discovery, thousands to hundreds of thousands of compounds are screened in the hope of discovering active compounds. The evaluation of a single compound can cost from a few cents to several dollars depending upon the complexity of the assay. The initial “hits” are unlikely to be the final drugs. Complex evaluations are necessary and typically, the initial hit is modified atom-by-atom to improve important characteristics of the molecule. It is desirable to find compounds of several different structural classes. If multiple chemical classes can be found, they provide optional starting points for further optimization of activity, physical properties, tissue distribution, plasma half-life, toxicity, etc.

### BACKGROUND OF THE INVENTION

A first step in the process of determining features of compounds that are important for biological activity is describing the molecules in a manner that is both capable of being analyzed and relevant to the biological activity. A drug-like molecule is a small three dimensional object that is often represented by a two dimensional drawing. This two dimensional graph is subject to mathematical analysis and can give rise to numerical descriptors to characterize the molecule. Molecular weight is one such descriptor. There are many more. Ideally, the descriptors will contain relevant information and be few in number so that the subsequent analysis will not be too complex. To exemplify our methods we use a system of BCUT descriptors given by Pearlman and Smith, 1998, the entire disclosure of which is incorporated herein by reference, which are derived from a method of Burden, 1989, the entire dosclosrue of which is likewise incorporated herein by reference. These descriptors are eigenvalues from connectivity matrices derived from the molecular graph. Atom properties are placed along the diagonal of a square matrix, a property for each non-hydrogen atom. Off diagonal elements measure the degree of connectivity between two atoms. The atomic properties can be size, atomic number, charge, etc. Since eigenvalues are matrix invariants, these numbers measure properties of the molecular graph. Also, since the properties on the diagonal measure important atomic properties, these numbers measure important molecular properties. There are 67 BCUT descriptors described by Pearlman and Smith, 1998.

A key challenge in statistical modeling of data of this sort is that the potent compounds of different chemical classes can be acting in different ways: Different chemical descriptors and narrow ranges of those descriptors might be critical for the different mechanisms. A single mathematical model is unlikely to work well for all mechanisms. Another challenge is that the molecular descriptors (explanatory variables) are often highly correlated. This is the case for BCUT numbers. We describe and claim a cell-based analysis method that finds small regions of a high dimensional descriptor space where active compounds reside. The method selects compounds accounting for correlations in the data.

As stated above, one of the initial steps in drug discovery is the screening of many compounds, looking for compounds that show potential biological activity. In addition to finding active compounds among those screened, it would be very useful to know how to find additional active compounds without having to screen each compound individually. We might initially screen part of a collection and having the results for the compounds screened, we would like to know which of the unscreened compounds are likely to be active. To do this we need to analyze the initial high throughput screening data to find association rules linking biological activity to specific values of the compound descriptors. The difficulties are as follows:

1. Active compounds can act through different mechanisms.

2. The linking relationships between activity and molecular descriptors are often non-linear. Thresholds often exist. There may be complex interactions among the descriptors. There is typically high correlation among descriptors.

3. Common statistical analysis methods such as linear additive models, generalized additive models, and neural nets can be ineffective.

The statistical analysis method of the present invention overcomes these problems. It finds small regions of a high dimensional space where active compounds reside. Untested compounds that live in these regions have a high chance of being active. Our method can also improve prediction accuracy over recursive partitioning.

The invention presents several novel features as follows.

1. Slicing low dimensional projections into bins and focusing the analysis on the resulting cells.

2. There are a very large number of resulting cells so there is a statistical risk that several active compounds will appear in a cell by chance alone. We compute the probability of finding k or more active compounds among the n compounds in a cell. This probability can be adjusted to take into account the number of cells examined, the Bonferroni adjustment. We improve this adjustment by taking into account the number of compounds in a cell. Statistical significance is not possible unless there are enough compounds in a cell. This results in a smaller adjustment and more statistical power to declare a cell active.

3. It is well-known that active compounds tend to reside in very small parts of chemical space. This analysis method is designed to find small active regions. The method is designed to work even if there are multiple mechanisms for activity.

4. It is possible that the cells created by the original slicing of the low dimensional projects will not be centered on the active regions. We shift the cells and thereby find additional active cells.

5. Information, active cells, from the different low dimensional projects can be combined to provide better predictions of the activity of untested compounds. This combining of information uses data from correlated variables and also from other dimensions.

### SUMMARY OF THE INVENTION

1. Divide data into Training and Validation sets as follows. Sort the data set by potency and place, at random, one of each consecutive pair into the two data sets.

2. Use all 67 BCUTs and focus on low-D subspaces. This leads to 50,183 ID/2D/3D subspaces.

3. Apply the data-driven hybrid binning method to bin all subspaces using 729 cells/subspace, leading to roughly 15-20 compounds per cell.

4. Create shifted cells from the original cells.

5. Training set: Compute summary statistics for each cell and note the cells with at least 3 hits and a hit rate of 20% or higher.

6. Repeat Step 5 but randomly re-order Y (random permutation). Define ‘cut-off’ for the good cells as the 10^{th} best value under random permutation. We use the 10^{th} value instead of the most extreme value (from millions of cells examined).

7. Training set: Define good cells as cells with better value than the cut-off value in Step 6.

8. Training set: Rank the good cells by the cell selection criteria (e.g. PValue, HR, BHRLow, MeanY, MLow, NHRLow).

9. Training set: Assign a score to each of the good cells using the score functions.

10. Validation set: Select validation compounds based on the top cells (Top cells method).

11. Validation set: Compute score for each validation compound and rank these compounds by their scores (Frequency selection/Weighted Score Selection).

12. Validation set: Select the highest ranked compounds based on these selection methods and evaluate their corresponding validation hit rates.

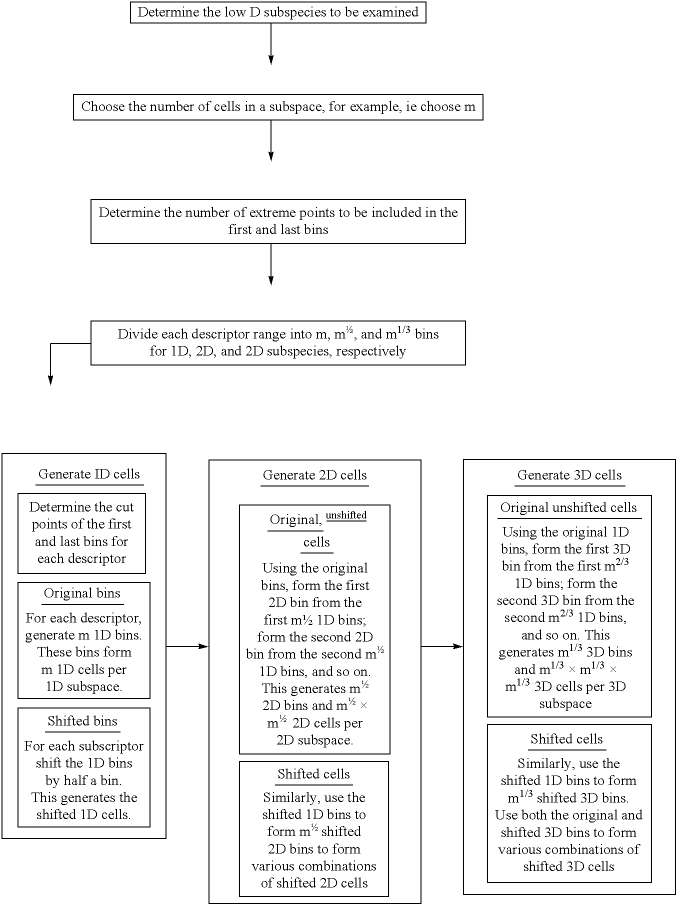

Method of dividing a high dimensional space into a finite number of low dimensional cells, including the “shifted” cells.

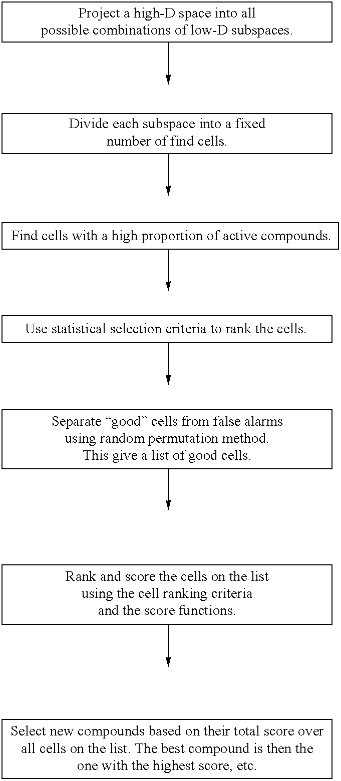

Method of identifying compounds for screening

### BRIEF DESCRIPTION OF THE DRAWINGS

FIGS. 1a and 1b are graphs illustrating clusters of active compounds from two different mechanisms for Cluster Significant Analysis.

FIGS. 2a and 2b are graphs illustrating why recursive partitioning might work or not work.

FIG. 3 is a graph illustrating the concept of shifted bins and overlapping cells.

FIG. 4 is a graph illustrating the overlapping of shifted cells, forming an active region.

FIG. 5 is a graph illustrating the performance of the frequency selection method based on the NCI compounds.

FIG. 6 is a graph illustrating the performance of the frequency selection method based on the Core98 compounds.

References

The following publications are included in the disclosure of this specification, each being incorporated in their entirety into this specification by reference.

Bayley, M. J. and Willett, P. (1999) Binning schemes for partition-based compound selection. J of Molecular Graphics and Modeling 17, 10-18.

Burden, F. R. (1989) Molecular Identification Number for Substructure Searches. Journal of Chemical Information and Computer Sciences 29, 225-227.

Hawkins, D. M., Young, S. S., and Rusinko, A. (1997) Analysis of a large structure-activity data set using recursive partitioning. Quant. Structure-Activity Relationship 16, 296-302.

Higgs, R. E., Bemis, K. G., Watson, I. A. and Wike, J. H. (1997) Experimental Designs for Selecting Molecules from Large Chemical Databases. Journal of Chemical Information and Computer Sciences 37, 861-870.

Jones-Hertzog, D. K., Mukhopadhyay, P., Keefer, C., and Young, S. S. (2000) Use of Recursive Patitioning in the Sequential Screening of G-protein Coupled Receptors. paper submitted.

Lam, R. L. H., Welch, W. J., and Young, S. S. (2000) Uniform Coverage Designs for Molecule Selection. paper submitted to Technometrics.

McFarland, J. W. and Gans, D. J. (1986) On the Significance of Clusters in the Graphical Display of Structure-Activity Data. Journal of Medicinal Chemistry 29, 505-514.

Miller, R. G. (1981) Simultaneous Statistical Inference. 2^{nd} Ed. Springer-Verlag, New York

Pearlman, R. S. and Smith, K. M. (1998) Novel software tools for chemical diversity. Perspect. Drug Discovery Design 09/10/11 339-353.

Rusinko, A, III, Farmen, M. W., Lambert., C. G., Brown, P. L., Young, S. S. Analysis of a large structure/biological activity data set using recursive partitioning. Journal of Chemical Information and Computer Sciences 1999, 38, 1017-1026.

### DETAILED DESCRIPTION OF THE PREFERRED EMBODIMENTS OF THE INVENTION

Objects are described with continuous descriptors, e.g. for compounds one of the descriptors could be molecular weight. Typically there are ten or so numerical descriptors for each object.

BCUT molecular descriptors, or any generalization of them, are useful molecular descriptors when used in our analysis method. An atom-based property is used on the diagonal of the BCUT molecular descriptor matrix. This matrix has real elements and is symmetrical. An atom-to-atom distance measure, through bond or through space, is used for the off-diagonal elements. A relative weighting of the diagonal to off-diagonal is used, typically anywhere between ten to one and forty to one. The molecular descriptors are determined by computing the eigenvalues of the molecular matrix. The BCUT descriptors of Pearlhman and Smith are acceptable molecular descriptors. (NB: Pearlman and Smith, 1998, teach against the use of BCUT numbers for quantitative structure activity analysis.)

We use low-dimension projections of the chemical space, typically all 1D, 2D, and 3D projections. We divide each subspace into non-overlapping bins (or cells). In the case of chemistry problems we typically place a fixed percent of the distribution into the first and last bins and divide the remaining range into fixed width bins. We keep the number of bins constant in each subspace. So if the ID projections are divided into 64 bins, then the 2D projections are divided into 8×8=64 bins as well. To obtain statistically dependable estimate of activity in a bin requires multiple compounds in a bin. If the activity of a compound is measured as binomial (active/inactive, 1/0), then ten to twenty compounds are needed per cell. If the activity of a compound is measured as continuous, then five to ten compounds are needed in a cell. Bin width can be determined by adjusting the number of cuts to give these average sample sizes within a bin.

The active compounds may not center in the bins as they are initially chosen so the frame of reference can be shifted half a bin over, or half a bin up, or both. The activity of an individual molecule may be measured as a binomial variable, active/inactive, 1/0, or as a continuous measurement, e.g. percent binding. The cells are ranked by their level of activity. This ranking can be governed by any of several methods. For binomial activity, cells can be ranked by hit rate, x/n, P-value of x out of n active, statistical lower bound on hit rate, etc. For continuous activity, cells can be ranked by average activity, statistical lower bound on average activity, etc.

Once the cells have been ranked for activity, we determine a cut point where cells above the cut point are declared active and those below are declared inactive. The cut point is determined via simulation. The observed compound potency values are assigned at random to the compounds, i.e. the potency values are permuted. The entire analysis procedure is repeated on the permutation data set. The potency values are permuted again and the entire analysis is repeated again. Many repeats of this process allow the estimation of the distribution of the compound ranking under the assumption that the descriptors have no effect on the activity of the compounds. The cut point for the evaluation of the observed, ranked cells is taken to be a value that cuts off a small proportion of this distribution; five percent, one percent, and one tenth of a percent are typical values.

Active cells are useful for the prediction of activity of untested compounds. The activity of an untested compound can be predicted from the activity of a cell that it fits into. A compound can fit into more than one cell as cells can be determined by common variables and variables can be correlated. We can score and rank a set of untested compounds by using the active cells determined in the previous step in any of a number of ways. a. Compounds that fall into the first active cell are taken first, that fall into the second active cell are taken next, etc. b. A compound can be given a score equal to the number of active cells it falls into. Untested compounds are then ranked by their scores. c. Each active cell can be given a weight and the compound score is the sum of the product of the cell weight over the selected cells. Untested compounds are ranked by their compound scores.

The preferred embodiment of the method described here can be applied to both continuous and discrete responses. For illustration, a data set with continuous activity outcome (Core98) and a data set with binary activity outcome (NCI) are included.

### EXAMPLE 1

Core98 Molecular Data (Continuous Response)

Biological activity scores were obtained on a chemical data set, Core98, comprising 23,056 compounds. Core98 is a chemical data set from the Glaxo Wellcome collection. Activity was measured as % Inhibition and theoretically should range from 0 to 100 with more potent compounds having higher scores. Biological and assay variations can give rise to observations outside the 0-100 range. Typically, only about 0.5% to 2% of screening compounds are rated as potent compounds. The compounds are described by sixty-seven BCUT numbers. These sixty-seven continuous descriptors measure molecular bonding patterns, and atomic properties such as surface area, charge, hydrogen-bond donor and acceptor ability. We found that the sixty-seven BCUT descriptors are highly correlated. These correlations are high for at least two reasons. Scientists often devise descriptors that measure the same general property of a compound. Also, chemical compounds, including those in Core98, are generally made for a purpose; once a good compound is made, similar additional compounds are made in chemical lead optimization programs, which are well known to those or ordinary skill in the art of pharmaceutical discovery and development. Both of these situations can lead to strong correlations in chemical data sets.

### EXAMPLE 2

NCI Molecular Data (Binary Response)

The NCI chemical database can be obtained from the web site http://dtp.nci.nih.gov/docs/aids/aids_data.html. When we downloaded the data in May 1999, there were about thirty-two thousand compounds in the NCI DTP AIDS antiviral screen database. Some of these were removed as their descriptors could not be computed, leaving about thirty thousand unique molecules. Like the Core98 data, the same set of sixty-seven BCUT descriptors were computed for the NCI data. However, unlike the Core98 data where the response is continuous, the NCI compounds are classified as moderately active, confirmed active, or inactive. The first two classifications are combined as ‘active’.

Statistical Analysis Methods

Here we describe two statistical analysis methods commonly used for chemical data sets.

A. Cluster Significance Analysis

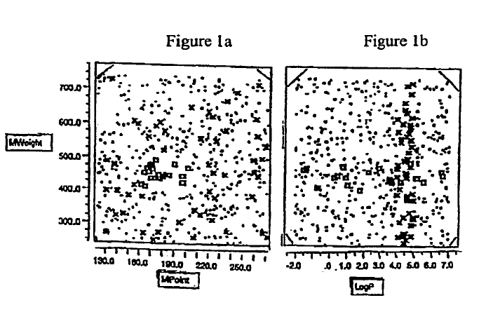

Cluster significance analysis (CSA), McFarland and Gans (1986), the entire disclosure of which is incorporated herein by reference, aims to find embedded regions of activity in a high dimensional chemical space. If for example, active compounds have a molecular weight between 400 and 500 and a melting point between 160 and 205 degrees C. If compounds that range in molecular weight from 250 to 750 and melting point from 100 to 300 degrees C. are tested, then simple statistical analysis methods, linear regression, can miss finding the relationship. A simple plot of the data shows the cluster of active compounds (squares in FIG. 1a). CSA computes the average distance between active compounds in a subspace of the high dimensional space and compares that distance to the average distance of an equal number of randomly selected inactive compounds. If the actives are clustered more tightly, then that is evidence that the dimensions where the actives are clustered are the descriptors that are important for activity. If the descriptors are compared, two at a time and the active compounds are clustered closely together only in the subspace of molecular weight×melting point, that would imply that these two descriptors are important. CSA tacitly assumes that there is only one class of active compounds.

If there is a second class of compounds acting through different mechanism the following analysis applies. For these compounds, there is biological activity if the octonal and water partition coefficient, termed logP, is between 4 and 5 (drugs typically range between −2 and +6). If only these compounds were plotted looking at all 1D and 2D plots, it would be found that the active compounds clustered in the 1D projection along the logP axis (crosses in FIG. 1b). But with large heterogeneous data sets it is seldom the case that those compounds are acting through one mechanism. If we plot the compounds from both mechanisms together in 2D, molecular weight×melting point, we see the second mechanism compounds (crosses in FIG. 1a) scattered across the 2D landscape. The creatures from the 3^{rd} dimension have come into the first two dimensions and corrupted our ability to clearly see the compounds following the first mechanism. So CSA, though it solves the problem of an imbedded region of activity for one mechanism is, under prior art methods, expected to fail for multiple mechanisms, heterogeneous data sets.

A synthetic data set is instructive of the method and the potential problems. Here there are plotted 2D scatter graphs, molecular weight versus melting point and molecular weight versus LogP. Each dot represents a compound. There are two active classes of compounds represented by Squares and Crosses in FIGS. 1a and 1b. Class I compounds (squares) are active and require that molecular weight be between 400 and 500 and that the melting point be between 160 and 205 degrees C. Note the concentration of active compounds within these ranges. Class II compounds (crosses) are active and require that the LogP be between the ranges 4.0 and 5.0. The CSA algorithm is going to have trouble. Although Class I active compounds concentrated in a small 2D region, the CSA will consider all the active compounds and not find the concentration. It will wrongly conclude that the pair of variables molecular weight and melting point are jointly not important. Examination of the 2D molecular weight versus LogP scatter graph shows that 1D LogP is important. But the CSA algorithm will not find this relationship as Class I compounds spread across the LogP range. The CSA presumes that there is only one class of active compounds and that they will concentrate in a small region of a subspace. If there are two active classes, then the CSA algorithm can be confused. In this synthetic example, active creatures from the 3^{rd} dimension contaminate dimensions one and two.

B. Recursive Partitioning Approach

The analysis of heterogeneous data sets is difficult. Heterogeneous data sets have more than one mechanism that gives rise to active compounds. Some of the compounds are active for one reason and others are active for other reasons. Many statistical methods are not expected to be successful with heterogeneous data sets. Recursive partitioning (RP) is a statistical method that can be successful with multiple mechanisms, Hawkins et al. (1997), Rusinko, et al. (1999), the entire disclosure of which is incorporated herein by reference. RP selects a descriptor and partitions the data based on one descriptor into two or more groups that are more homogeneous. Each daughter group is partitioned in turn until the groups are judged homogeneous or some minimum sample size is reached. This separation of the data into smaller data sets can separate the components of a mixture into separate groups where only a single mechanism is operable.

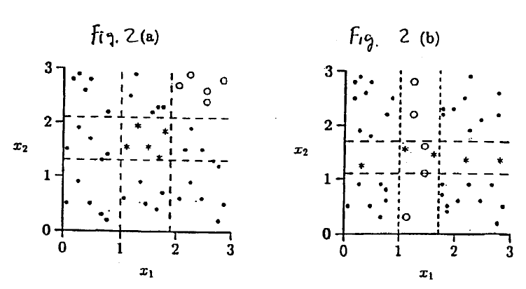

As successful as RP has been for the analysis of HTS data sets, Jones-Hertzog et al. (2000), there are a number of possible problems. First, this approach selects one descriptor at a time to split the data set. But a single descriptor may not provide adequate information for the splitting process. In addition, when the descriptors are highly correlated, selecting one descriptor will likely lead to deselecting several others. The second problem relates to multiple mechanisms when some of the active regions are near or overlapping each other. FIG. 2 illustrates configurations of active compounds from two mechanisms (the asterisks and circles) and inactive compounds (the dots) and the splits recursive partitioning (the dashed lines) might make when there were only one mechanism. x₁ and x₂ are some arbitrarily chosen chemical descriptors critical for the biological activity. In FIG. 2a, recursive partitioning will split on x₁ and x₂ around 1 and find one big region comprising both the two active regions for the 2 mechanisms and two irrelevant regions on the side. This illustrates that partitioning one variable at a time can be ineffective. In FIG. 2b, recursive partitioning might split twice on either x₁ and x₂, finding one of the two mechanisms. Partitioning reduces the sample size accordingly, and it would be difficult to separate the remaining active compounds from the inactive compounds. The third problem relates to the number of splits. Binary splits are often used in recursive partitioning. Problems can result if the activity pattern is inactive-active-inactive as, however the single cut point is chosen, actives will be combined with inactives. It is important to keep the following observation in mind: two compounds must have fairly close values of all critical descriptors for similar biological activity (McFarland and Gans, 1986, the entire disclosure of which is incorporated herein by reference) when there is a single mechanism. This means that partitions have to be narrow, and in several dimensions simultaneously, if all molecules from a partition are to have similar activity.

Cell-Based Analysis of Large Data Sets

The present invention introduces a cell-based analysis method that first identifies good regions (cells) in a high-dimensional descriptor space and then uses the information about good regions to score new compounds and prioritize them for testing.

Identifying good regions of the descriptor space involves three steps: (1) project a high-D space into all possible combinations of low-D subspaces and divide each subspace into cells; (2) find active cells (regions); and (3) refine the active cells. We use the data-driven binning method described by Lam et al. (2000), the entire disclosure of which is incorporated herein by reference, to generate the low-D subspaces and cells in these subspaces. Good cells (those with a high proportion of active compounds) are identified using several statistical selection criteria, based primarily on the hit rate in a cell and/or its reliability. To adjust or refine the original cell boundaries, we shift cells in each dimension of the subspace and hence identify good cells missed by the original binning. These techniques are described in further detail below in the section entitled “Identifying Good Cells”.

The good cells are then used to score and select new compounds with the best chance of being active. New compounds appearing in the most highly ranked cells or frequently amongst the good cells are promising candidates for testing. Alternatively, new compounds may be scored using one or more of the criteria used for cell selection, and these scores can be used for ranking the compounds. Details of the methods for selecting new compounds are provided in Section 6.

Identifying Good Cells

Binning the Descriptor Space into 1D/2D/3D Cells.

The advantage of dividing a space into cells is that a number of methods can be developed to identify good cells, i.e., those with a high proportion of active compounds. We now review some methods for dividing a high-dimensional space into many fine, low dimensional cells.

In a conventional cell-based method, the range for each of the descriptors is subdivided into m bins of equal size. With the 67 BCUT descriptors, we would have m⁶⁷ cells. Even with m=2, there are 2⁶⁷ cells generated, most of which are empty even for the largest ever-existing chemical database. There would be more cells than data points and in addition, most compounds will be densely clustered. If most of the compounds are concentrated into relatively few cells, it becomes difficult or impossible to separate active and inactive regions.

Following Lam et al. (2000), we focus our attention on low-dimensional subspaces, typically all 1-D, 2-D, and 3-D subspaces. We keep the number of cells constant over each subspace, avoiding the exponential increase in the number of cells with dimension. To avoid empty cells caused by the scarcity of molecules towards the limits of a descriptor's range, we adopt a data-driven hybrid binning method that makes bins larger towards the extremes. For more details in binning a high dimensional space into low dimensional cells, see sections 4.1 (Forming Cells) and 4.2 (Data-Driven Binning) in Lam et al. (2000). Briefly, cells are created as follows. Initially, we divide each descriptor into m bins. For each descriptor, these bins are immediately the cells for its 1-D subspace. To form the cells for a given 2-D subspace, amalgamate the bins of each of its dimensions into m^{1/2} bins. There are m^{1/2}×m^{1/2}=m 2-D cells from combining these larger bins. Similarly, to form 3-D cells, we amalgamate each dimension's 1-D bins into m^{1/3} bins; these are combined to give m^{1/3}×m^{1/3}×m^{1/3}=m 3-D cells. Thus, all subspaces, whether 1-D, 2-D, or 3-D, have the same number of cells. To generate integer numbers of bins, it is convenient if m is an integer raised to the power of 6, e.g., 2⁶=64 or 3⁶=729. We give further guidance below on choosing m.

With k descriptors, there are

$$( \begin{matrix}k \\ 1\end{matrix} ) + ( \begin{matrix}k \\ 2\end{matrix} ) + ( \begin{matrix}k \\ 3\end{matrix} ) = \frac{5}{6} k + \frac{1}{6} {k}^{3}$$

1-D, 2-D, and 3-D subspaces in total. For every subspace, a molecule is in one and only one cell. The goal is to find a set of cells in which there are many active compounds and a high proportion of active compounds.

How large should the bin size be? Cells formed from large bins may contain more than one class of compounds. Moreover, if only part of the cell is good, active compounds will be diluted by inactive compounds and the cell may be deemed inactive. (Two compounds must have fairly close values of all critical descriptors for similar biological activity.) On the other hand, cells formed by very fine bins may not contain all the compounds in the same class. Furthermore, very small cells will tend to have very few compounds and there will be little information to assess the quality of the cell. We make the bins fine, but not too fine given N, the number of assayed compounds. For reliable assessment of each cell's hit rate, we would like about 10 to 20 compounds per cell. This suggests that the number of cells per subspace should be about N/20 to N/10. When cells are shifted (described in Section 5.4), additional active regions missed by the original binning may be found.

Intra-subspace cells (not including the shifted cells) within a subspace, are mutually exclusive and cover different sets of compounds. On the other hand, inter-subspace cells, cells from different subspaces, may overlap and can cover the same set of compounds. The compound-selection method described in Section 6 takes advantage of the overlapping in inter-subspace cells and the correlations between descriptor variables.

Ranking Cells

A natural first choice for the identification of active cells is to compute the proportion of all the compounds in the cell that are active (the observed hit rate) and then rank the cells by these proportions. Cells with a high proportion of active compounds are declared active. The main problem with this method is that it favors cells that happen to have a small number of compounds. Consider two cells with 2/2 and 19/20 active compounds, respectively. The first has a hit rate of 100%, but this is based on two compounds, a very small sample. The 95% hit rate for the second cell is based on 20 compounds, and is much more reliable. Therefore, several of our criteria for cell selection take into account the statistical variability from sampling as well as the raw hit rate.

Different types of activity data are possible, and this is reflected in the criteria. First, compounds may be assayed as either “Active” or “Inactive”. The p-value (PValue), hit rate (HR), and binomial hit rate lower confidence limit (BHRLow) criteria below are relevant to data of this type. Secondly, an assay may give a numerical value Y (e.g., percentage inhibition) for activity. In this case, we have the mean activity score (MeanY), lower confidence interval for mean Y (MLow), and normal hit rate lower confidence limit (NHRLow) below. Data of this second type may be converted to the first type by defining “Active” as Y>c for some cut-off c, allowing all criteria to be used.

P-Value (PValue)

Let N be the number of compounds in a data set (e.g., 11528 compounds in the Core98 training set), and let A be the number of active compounds in the data set (e.g., 100 active compounds). Consider a given cell in a given subspace. It has n compounds, of which x are active.

Suppose the A active compounds are distributed such that they fall in or outside the given cell at random. Under this statistical null hypothesis, the probability of observing x actives out of n compounds is given by the hypergeometric distribution:

$$Prob ( x ; n , A , N ) = \frac{( \begin{matrix}A \\ x\end{matrix} ) ( \begin{matrix}N - A \\ n - x\end{matrix} )}{( \begin{matrix}N \\ n\end{matrix} )}$$

The p-value is the probability of having at least x active compounds out of n:

p-value=Prob(X≧x|n compounds)

$$\begin{matrix}p \text{-} value = Prob ( X \geq x \vert n compounds ) \\ = {\sum }_{i = x}^{min ( A , n )} \frac{( \begin{matrix}A \\ i\end{matrix} ) ( \begin{matrix}N - A \\ n - i\end{matrix} )}{( \begin{matrix}N \\ n\end{matrix} )} = 1 - {\sum }_{i = 0}^{x - 1} \frac{( \begin{matrix}A \\ i\end{matrix} ) ( \begin{matrix}N - A \\ n - i\end{matrix} )}{( \begin{matrix}N \\ n\end{matrix} )} .\end{matrix}$$

If the p-value is small, there is little chance of seeing x or more active compounds out of n. Therefore, small P-values provide the most evidence against the null hypothesis of random allocation of actives in/outside the cell (and hence most evidence that the number of actives in the cell is better than chance). The P-value is computed for all cells and the cell with the smallest P-value is the top-ranked cell, etc.

One problem with the p-value approach is that it tends to pick cells with large numbers of compounds even if they have fairly low hit rates. For example, 15 actives out of 174 gives p<0.0001 but 4 out of 4 gives p=0.0014. The statistical evidence is stronger in the first case because of the larger sample size, even though the hit rate is much lower. To tilt the process toward higher hit rates when screening new compounds, one may rank by p-value only those cells with a hit rate of, say, at least 30%.

Hit Rate (HR)

In the above notation, the hit rate for a cell is x/n. It ignores the increased reliability from a larger sample size. For example, 1/1 gives a 100% hit rate but 9/10 gives a 90% hit rate; the cell with 9/10 is more promising. A simple way to solve this problem is to consider only cells with several active compounds. Another criterion, such as p-value or mean activity score, can be used to break ties when two cells have the same hit rate.

Binomial Hit Rate Lower Confidence Limit (BHRLow)

One can obtain an exact lower confidence limit on the hit rate for new compounds based on the binomial distribution. For the many possible compounds that would fall in a given cell, suppose that a proportion h are active, i.e., h is the hit rate. Assuming that the n compounds in the cell that have been assayed are a random sample of all the cell's possible compounds, the number observed to be active follows a binomial distribution with n trials and probability h. The smallest value of h such that Prob(X≧x|h, n)=0.05 is the 95% binomial hit rate lower confidence limit (BHRlow). It considers both the hit rate and its variability. BHRlow seems to be a better predictor for validation hit rates than either the hit rate or p-value. The BHRlow approach is effective when the cell size is large. When there are only few compounds in each cell, BHRlow may become insensitive and tends to pick cells or regions with smaller number of hits and with very high hit rate. For example,

BHRlow (3/3)=0.3684 but BHRlow (8/15)=0.2999

and

BHRlow (6/6)=0.6070 but BHRlow (12/15)=0.5602.

One can avoid this problem by assuming that a good cell must have several active compounds.

Mean Activity Score (MeanY)

When a numerical assay value, Y, is available, the mean over all compounds in a cell gives the mean activity score (MeanY). Because it is easier by chance to obtain a high mean from fewer compounds than from more compounds, MeanY tends to pick cells with few compounds (e.g., two compounds with high activity values). As with HR, one can avoid this problem by considering only those cells with several active compounds.

Lower Confidence Limit for Mean Y (MLow)

Analogous to BHRLow, with a numerical assay value, Y, one can use the lower confidence limit for the mean of the distribution giving the Y values (MLow), based on an assumption of sampling from the normal distribution. MLow considers both the observed mean and the variability and is defined as

MLow=\overline{Y}−\hat{σ}/\sqrt{n}×t(d,0.95),

where \hat{σ} is an estimate based on d degrees of freedom of the standard deviation of the Y distribution within the cell, and t(d, 0.95) denotes the 95% quantile of the t distribution with d degrees of freedom.

We use a common value of \hat{σ} for all cells within a subspace. For a given subspace, it is computed by pooling the sample variances over all cells:

$${\hat{σ}}^{2} = \frac{\sum ( {n}_{i} - 1 ) {s}_{i}^{2}}{\sum ( {n}_{i} - 1 )} ,$$

where sᵢ² is the sample variance for cell i, and cell i has nᵢ compounds.

MLow seems to perform better than Mean Y in ranking the cells.

Normal Hit Rate Lower Confidence Limit (NHRLow)

With a numerical measure of activity, Y, and a cut-off for activity, c, one can derive a lower confidence limit for the probability Prob(Y>c) based on the normal distribution. This criterion is called NHRLow.

Assuming that the Y values are randomly sampled from a normal distribution with mean μ and variance σ², the exact NHRLow is determined as follows.

$$Pr ( Y > c ) = 1 - Pr ( Y \leq c ) = 1 - Pr ( \frac{Y - μ}{σ} \leq \frac{c - μ}{σ} ) = 1 - Φ ( \frac{c - μ}{σ} ) = Φ ( \frac{μ - c}{σ} ) ,$$

where Φ is the standard normal cumulative distribution function.

If σ is known (we can obtain a good estimate of σ by pooling the sample variances over all cells in a subspace, as above), then we can estimate Φ by

$$\hat{Φ} = Φ ( \frac{\overline{Y} - c}{σ} ) ,$$

where \overline{Y} is the average Y value for the n compounds in the cell. Let

$$Z = \frac{μ - c}{σ} ,$$

which we estimate by

$$\hat{Z} = \frac{\overline{Y} - c}{σ} .$$

We have

$$E ( \hat{Z} ) = \frac{μ - c}{σ}$$

and

$$Var ( \hat{Z} ) = \frac{{σ}^{2}}{n} \frac{1}{{σ}^{2}} = \frac{1}{n} .$$

Therefore,

$$\hat{Z} ∼ N ( \frac{μ - c}{σ} , \frac{1}{n} ) and Pr ( \frac{\hat{Z} - \frac{μ - c}{σ}}{1 / \sqrt{n}} < {Z}_{.95} ) = 0.95 ,$$

where Z_{0.95} is the 95% quantile of the standard normal distribution.

Rearrangement of the inequality gives

$$P r ( {Z}_{L} < \frac{μ - c}{σ} ) = 0.95 , where {Z}_{L} = \hat{Z} - \frac{{Z}_{.95}}{\sqrt{n}} .$$

The 95% confidence interval (CI) for

$$\frac{μ - c}{σ}$$

is (Z_{L},∞) and the corresponding 95% CI for

$$Φ ( \frac{μ - c}{σ} )$$

is (Φ(Z_{L}), 1) since Φ is a monotonic increasing function.

Therefore,

$$NHRLow = Φ ( {Z}_{L} ) = Φ ( \frac{\overline{Y} - c}{σ} - \frac{{Z}_{.95}}{\sqrt{n}} ) .$$

Relationships Between the Criteria

If a numerical measure of activity is available, all six criteria can be used. The cut-point c for activity (hit) is used as follows. For PValue, HR and BHRLow, c is used to convert the data to “Active”/“Inactive” before they are computed. Both MeanY and MLow ignore c. For NHRLow, the y distribution is modeled and c is used at the end to determine NHRLow.

Multiple Testing

With 67 descriptors, there are a total of 50,183 1D/2D/3D subspaces. If each subspace is divided into 729 cells, there are 36,583,407 cells. With so many cells, it is possible that by chance alone we will see cells with moderate activity.

Consider the p-value criterion. To adjust it for the total number of cells examined, C, we simply multiply the p-value by C. This is the Bonferroni correction, Miller (1981), the entire disclosure of which is incorporated herein by reference. In the training data, a cell is said to be a good cell if the Bonferroni adjusted p-value is small (say<0.05).

The Bonferroni correction tends to over-correct. In the Core98 example with 67 BCUTs and 729 cells/subspaces, we examined a total of 36,583,407 cells, of which only 19,010,520 cells have at least three compounds. Cells with less than three compounds cannot be deemed active because of the small sample size. Thus, we adjust the p-value by multiplying by the number of cells with at least three compounds.

We can also impose a minimum hit rate to define the cells relevant for correction. For example, in the Core98 training data, if we only consider cells with at least three active compounds and a hit rate of at least 50%, then there are only 3,144 cells if the threshold for activity is set so that 0.5% of all compounds are active.

Probably the best way of addressing the multiple testing problem is to define the cut-off between active and inactive cells using a random permutation of the assay values. We randomly reorder the Active/Inactive or Y values in the training data. If p-value is the criterion for ranking cells, we set the cutoff as the smallest p-value obtained. Under random permutation of the data, no cells should be identified as good cells and the smallest p-value is just due to chance. For the actual data (without permutation) we then use all cells with p-value smaller than this cut-off value. This permutation method can be used to set the cut-off value for any of the cell-based selection methods.

Shifted Cells

The hybrid binning method generates non-overlapping cells within a subspace. We call these the original, unshifted cells. To allow for the fact that this binning may not be optimal, we also shift the original cells in the various dimensions to create overlapping cells. For example, in a 2D subspace, we generate four sets of cells: one set of original, unshifted cells, two sets of cells with only one dimension shifted by half a bin, and one set of cells with both dimensions shifted half a bin.

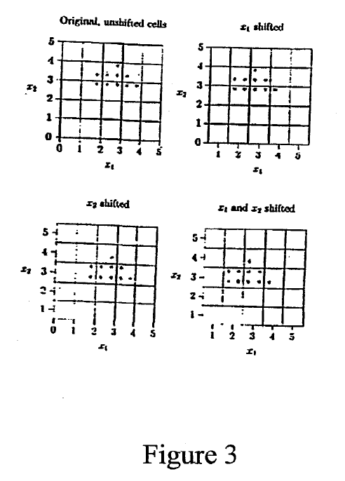

This is illustrated in FIG. 3, which shows the locations of 10 active compounds in the subspace formed by two descriptors, x₁ and x₂. To form 2D cells, the range of each descriptor is divided into five bins. The original, unshifted cells are shown in the top left graph of FIG. 3. To create the shifted cells, first we shift the x₁ bins by half a bin but keep the x₂ bins fixed (see the top right graph). Next we shift the x₂ bins by half a bin but keep the x₁ bins fixed (see the bottom left graph). Finally, we shift both x₁ and x₂ by half a bin (see the bottom right graph).

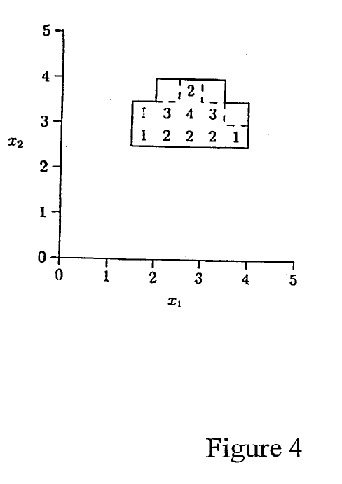

If a good cell has to have at least three active compounds, there is one active cell in each of the two top graphs and there are two active cells in each of the two bottom graphs. The region formed by these overlapping active cells is shown in FIG. 4. The counts are the number of times each active compound is selected by an active cell. The dashed lines show how the active region could be adjusted to exclude sub-regions with no actives. The shifted cells provide an efficient method for refining active regions.

We also investigated several methods for re-sizing an active region. However, we found that re-sizing a cell around an original, active cell was not as effective and efficient as shifting cells.

Selection of New Compounds

We present three selection methods for choosing new compounds for biological screening: ‘Top Cells Selection’, ‘Frequency Selection’ and ‘Weighted Score Selection.’

Top Cells Selection

This method first ranks cells according to one of the cell selection criteria described earlier. In a database of new, unassayed compounds, it then chooses all the compounds falling in the best cell, then all those in the second best cell, and so on until the desired number of compounds to be tested is reached or until there are no good cells remaining from the initial cell-based analysis.

Good cells from different subspaces may overlap because of shared descriptors. Thus, a new compound may appear in several highly ranked cells. The top cells selection approach does not take this into account, lowering the hit rate in the validation set. The next method exploits the information from overlapping cells.

Frequency Selection

High correlation exists among the 67 descriptors, a property of the BCUTs. Thus, if a new compound falls in a highly ranked cell, it is likely to fall within further highly ranked cells sharing correlated descriptors. The frequency selection method takes advantage of these correlations in the data. It selects compounds based on the frequency of occurrence in the list of good cells.

We rank the new compounds by the number of times they appear in the list of highly ranked cells. (The length of the list can be determined by the random permutation approach, for example.) The first compound selected for screening is the one occurring with the maximum frequency. The second compound selected has the second largest frequency, and so on. Compounds residing in several overlapping regions have the highest chance of being active, as information from many descriptors is being exploited. The frequency selection method greatly improves the validation hit rate for the first tens of compounds selected.

Weighted Score Selection

Instead of just counting the frequency of occurrence in the list of good cells, we can apply a weight function to the cells and select compounds based on their total weighted score over all cells on the list. The best compound is then the one with the highest score, etc.

The cell selection criteria described earlier can be adapted as weight functions. We could use the BHRlow value or −log(p-value) as weights, for example. These weight functions should have several desirable properties: (1) If the list of good cells is extended the relative weights of the cells in the original list should not change; (2) the weight function should be a smooth monotonic decreasing function of the cell's rank; and (3) the same weight should be assigned to cells rated equally by the cell selection criteria.

Performance Evaluation

In this section we evaluate the performance of the cell-based method using the Core98 and NCI data sets.

Dividing Data into Training and Validation Sets

For the purpose of demonstrating the validity of the new methods, we divide the original data into a training set and a validation set. We use the training data set (treated as screened compounds) to build models (i.e., find active regions) and the validation data set (treated as unscreened compounds) to evaluate prediction accuracy (i.e. verify if the activity in these regions remains high). In real applications we would use all the data to find active regions.

It is often useful to have more than one data set measuring the same biological activity to study, develop and exemplify a new statistical method. The first data set, the training set, can be used to calibrate the statistical prediction method. The second data set, the validation data set, can be used to test its effectiveness. If we develop a statistical prediction method using the training set, there is a risk that the method “memorizes the data” and that predictions based upon the training set are overly optimistic. If the method is used to predict the “hold out” or validation data set, then we get a more unbiased evaluation of the effectiveness of the statistical method. Of course, the real test is to apply the method on a completely independent data set.

Since there are only about 0.5% to 2% of screening compounds are rated as active, it is important to carefully allocate sufficient active compounds into both training and validation data sets such that good regions can be identified and validated. We sort the data set by the assay value and place, at random, one of each consecutive pair into the two data sets.

Evaluation Plan

Evaluation Steps

1. Divide data into Training and Validation sets as follows. Sort the data set by potency and place, at random, one of each consecutive pair into the two data sets.

2. Use all 67 BCUTs and focus on low-D subspaces. This leads to 50,183 1D/2D/3D subspaces.

3. Apply the data-driven hybrid binning method to bin all subspaces using 729 cells/subspace, leading to roughly 15-20 compounds per cell.

4. Create shifted cells from the original cells.

5. Training set: Compute summary statistics for each cell and note the cells with at least 3 hits and a hit rate of 20% or higher.

6. Repeat Step 5 but randomly re-order Y (random permutation). Define ‘cut-off’ for the good cells as the 10^{th} best value under random permutation. We use the 10^{th} value instead of the most extreme value (from millions of cells examined).

7. Training set: Define good cells as cells with better value than the cut-off value in Step 6.

8. Training set: Rank the good cells by the cell selection criteria (e.g. PValue, HR, BHRLow, MeanY, MLow, NHRLow).

9. Training set: Assign a score to each of the good cells using the score functions.

10. Validation set: Select validation compounds based on the top cells (Top cells method).

11. Validation set: Compute score for each validation compound and rank these compounds by their scores (Frequency selection/Weighted Score Selection).

12. Validation set: Select the highest ranked compounds based on these selection methods and evaluate their corresponding validation hit rates.

Evaluation Objective

We evaluated the performance of our cell-based analysis method using the 23056 Core98 compounds with biological assay Y11 and using the 29812 NCI compounds. The objective of this evaluation is to (1) determine if the new methods lead to higher hit rates than random selection, (2) assess the effect of the six cell selection criteria on hit rate, and (3) determine whether our cell selection method can find real active cells or false alarms.

For the purpose of evaluation, we define the Core98 compounds as active/inactive as follows. First, we define an active compound as a compound having an activity value 50 or higher. Of the 23056, only 103 (0.45%) compounds are active by this definition.

Secondly, we refer to the 0.45% as the population hit rate or the random hit rate. This hit rate gives us benchmark for the performance of our analysis method. If the hit rates based on the new method turn out to be many times higher than the random hit rate, then the new methods performs well.

Results

The NCI and Core98 compounds are divided into Training and Validation sets and are summarized below.

| | Training | Validation |
|---|---|---|
| | # actives/inactives | # actives/inactives |
| NCI(N = 29812) | 304/14602 | 304/14602 |
| Core98(N = 23056) | 51/11477 | 52/11476 |

We use the population hit rate (or random hit rate) from the validation data as benchmark. The results are divided into two parts. Part I describes good cells versus false alarms based on the training data and Part II describes hit rates obtained based on the validation data.

Good Cells Versus False Positives

P-Value Correction

To test whether our cell-based method would give false-positive results (i.e., declare cells good when they are not), the activity values were randomly re-assigned to the compounds. The same analysis was carried out several times as if the values were real, using various settings of random hit rate (0.5%, 1%, 2%) and bin sizes (729 and 4096 cells/subspaces). Using the p-value correction method described earlier, not even a single cell was declared good. On the other hand, hundreds to thousands of good cells were found using the real activity value. The over-correction approach addresses the false positive problem but is overly conservative.

Cut-Off for Good Cells

Random permutation test was performed to find the cut-off separating good cells from the rest for both the Core98 and NCI training data sets.

For the NCI compounds, we found that under random permutation, the 10^{th} best PValue and BHRLow values were 1.70*10⁻⁷ and 0.3684 and that with the real activity values, 301,156 cells had PValue<1.70*10⁻⁷ and 88,679 cells gave BHRLow>0.3684. The 10^{th} best HR under random permutation is 1.0 (3/3). As mentioned earlier, HR alone is not a useful criterion for defining a cut-off.

For the Core98 compounds, we found that under random permutation, the 10^{th} best PValue and BHRLow values were 1.25*10⁻⁶ and 0.0977 and that with the real activity values, 41,300 cells had PValue<1.25*10⁻⁶ and 43,650 cells gave BHRlow>0.0977. The 10^{th} best HR under random permutation is 0.3333 (3/9).

| | Under Random Permutation | | | Real Data | |
|---|---|---|---|---|---|
| | Cell value (#actives/#comps) | | | # cells with > value | |
| | | PValue | BHRLow | PValue | BHRLow |
| NCI | Best | 5.71E−8 (6/12) | 0.4729 (4/4) | 281,279 | 34,917 |
| | 10^{th} | 1.70E−7 (4/4) | 0.3684 (3/3) | 301,156 | 88,679 |
| | best | | | | |
| Core98 | Best | 1.64E−7 (4/12) | 0.1288 (3/7) | 21,843 | 24,612 |
| | 10^{th} | 1.25E−6 (4/19) | 0.0977 (3/9) | 41,300 | 43,650 |
| | best | | | | |

Alternatively, we can combine both the best 10^{th} PValue and BHRLow to define a ‘common’ cut-off. For the NCI compounds, there are 84,021 cells with PValue<1.70*10⁻⁷ and BHRLow>0.3684. For the Core98 compounds, there are 30,378 cells with PValue<1.25*10⁻⁶ and BHRlow>0.0977. With a common cut-off, HR can also be used to rank the good cells. Similar results are obtained using a common cut-off and thus are not included here.

Validation of Hit Rates

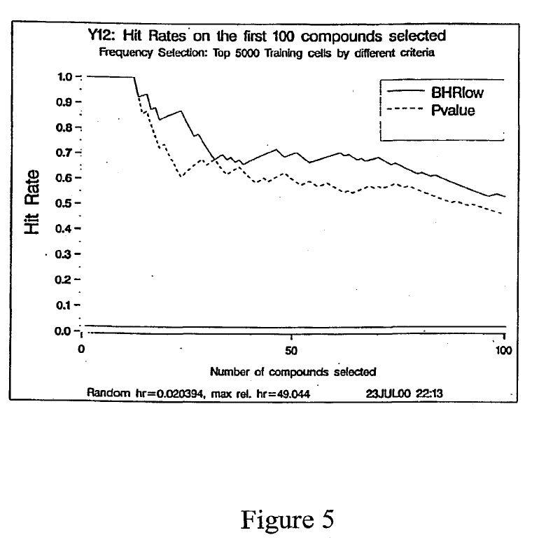

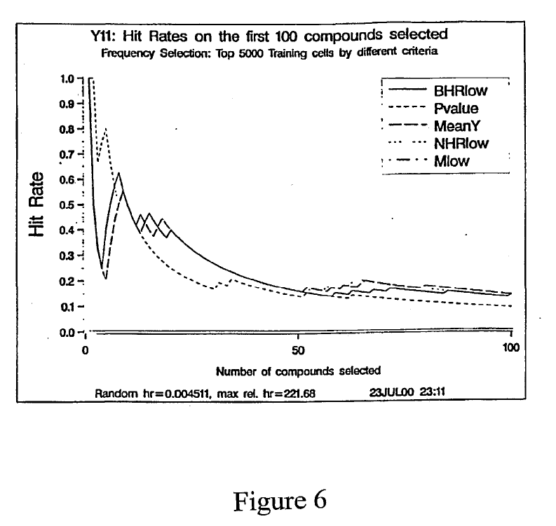

Compounds in the validation sets were selected based on the top 5000 cells using the weighted score selection method. The corresponding hit rates based on the different cell ranking criteria are shown in FIGS. 5 and 6 for the NCI and Core98 compounds, respectively. The cell-based analysis method, with any of the cell ranking criteria, leads to hit rate many times higher than the random hit rate (the horizontal line in the figures).

As the smallest p-value computed in SAS is 1×10⁻¹⁶, it was not possible to properly rank and score the cells using the PValue criterion in SAS. For example, with the NCI training data, 41,278 cells had PValue<1×10⁻¹⁶ which was set to zero by SAS. These 41,278 cells were given the same rank in SAS. Regardless, the frequency selection and the weighted score selection methods are robust.

Results

These results confirm that (1) the cell-based method is useful in identifying good cells, (2) many good cells were found, not false alarms, and (3) the BCUT descriptors are informative. Our cell-based analysis method leads to hit rates many times higher than the random hit rate. The Cell Selection method identified thousands of good regions. The top cells method selected the top active regions. The frequency selection method selected the most promising compounds with high hit rate.

Our cell-based analysis method handles the following statistical modeling issues:

1. Nonlinearities

2. Thresholds

3. Interactions

4. Multiple mechanisms

5. Highly correlated descriptors.

The foregoing is not an exhaustive list or description of all possible embodiments of the present invention, and all alternative embodiments that are possible with the use of equivalent steps, algorithms or materials that are acceptable to those of ordinary skill in the art are intended to be included within the scope of the claims below. It is noted further that in alternative embodiments of the present invention, any of the above methods are implemented by use of a suitable computer or other suitable processors and are additionally capable of partial or complete implementation by transfer of data over the international network of computer terminals, exchanges and servers (the Internet).

What is claimed is:

1. A cell-based analysis method that finds small regions of a high dimensional descriptor space comprising the steps of:
(a) projecting a high-D space into all possible combinations of low-D subspaces and dividing each said subspace into a corresponding regional cell;
(b) finding active regional cells; and
(c) refining the active cells.

2. A method of dividing a high dimensional space into a finite number of low dimensional cells, comprising the steps of:
(a) determining the low dimensional subspaces for examination;
(b) choosing the number of cells in a subspace (m);
(c) determining the number of extreme points to be included in the first bin and last bin;
(d) dividing each descriptor range into m bins for 1-D subspace, m^{1/2} bins for 2-D subspace, and m^{1/3} bins for 3-D subspace; and
(e) generating 1-D cells, 2-D cells, and 3-D cells.

3. A method of identifying compounds for screening, comprising the steps of:
(a) projecting a high dimensional space into all possible combinations of low dimensional space;
(b) dividing each subspace into a fixed number of fine cells;
(c) finding cells with a high proportion of active compounds;
(d) using a statistical selection criteria to rank the cells;
(e) determining a list of good cells by use of random permutation;
(f) ranking and scoring the cells on the list by use of a cell ranking criteria and a score function; and
(g) selecting new compounds based on a total score of compounds over all cells on the list.
